(19) United States
(12) Patent Application Publication
(10) Pub. No.: US 2004/0220488 A1
(43) Pub. Date: Nov. 4, 2004

(54) Method and apparatus for physiological data acquisition via sound input port of computing device

(76) Inventors: Andrey Vyshedskiy, Boston, MA (US); William Kania, Westborough, MA (US); Raymond Murphy, Wellesley, MA (US)

Correspondence Address: Andrey Vyshedskiy, Suite 4990, 1153 Centre St., Boston, MA, 02130, US

(21) Appl. No.: 10/694,910

(22) Filed: Oct. 29, 2003

Related U.S. Application Data
(60) Provisional application No. 60/466,242, filed on Apr. 29, 2003.

(51) Int. Cl. A61B 5/04; A61B 5/02

(52) U.S. Cl. 600/513; 600/528; 381/67

(57) ABSTRACT

A sound input port is ubiquitously present in many types of devices including PCs, PDAs, cell phones, land line phones, and voice recorders thereafter referred to as “computing devices”. A sound port allows data input into a computing device for further computation, visualization and data transmission. Unfortunately most computing devices only allow one channel of data acquisition via the sound port. Further, the acquired data are highpass filtered. A method of extending the signal range to very low frequencies and recording a plurality of data channels via a single sound port is disclosed here. This method uses amplitude modulation of carrier frequencies to create a composite signal. The composite signal is then transmitted into the computing device either via wire or wirelessly. Demodulation occurs in the computing device. In the preferred embodiment the audio signal from an electronic stethoscope and the amplitude modulated EKG are transmitted into a computer via a single microphone port. In an alternative embodiment physiological data from multiple sensors are transmitted into a computer via a single microphone port.

Priority is claimed by provisional application No. 60/466,242 filed on Apr. 29, 2003 entitled Method of data acquisition via microphone port of a computer.

### FIELD OF THE INVENTION

The invention relates to systems used for physiological data acquisition. It also relates to diagnostic systems.

### BACKGROUND OF THE INVENTION

A sound input port is ubiquitously present in many types of devices including PCs, PDAs, cell phones, land line phones, voice recorders, etc., hereafter referred to as computing devices. This sound input port can be primarily of 3 types: 1) a microphone port, 2) a line port, and 3) a wireless port (e.g. Bluetooth Headset). These ports are similar in their frequency characteristics with two notable differences. A line port is designed for stronger “line-level” signals with peak-to-peak amplitude of approximately 10V. Furthermore a line port does not supply bias DC voltage. A microphone port is designed to receive smaller signals with peak-to-peak amplitude of approximately 100 mV. In addition, a microphone port normally provides a bias DC voltage. Microphones lacking their own power supply rely on bias DC voltage for their power source. The invention disclosed herein can be used with either line, microphone, or wireless ports. Consequently, all sound input ports are hereafter referred to as “a microphone port” or “a sound port”.

A microphone port allows analog data input into computing devices for further computation, visualization and data transmission. Unfortunately most computing devices only allow one channel of data acquisition via a microphone port. Standard multiplexing methods for transmitting a plurality of data channels via a single channel do not work since the microphone port has a hardware lowpass filter. The invention disclosed herein does not use a multiplexing method. Rather, the invention uses amplitude modulation of a plurality of data channels to transmit the composite signal into a microphone port of a computing device. All signals can be demodulated in the computing device with no loss of data.

Devices for concurrent recording of two or more channels of physiological data are well known. U.S. Pat. Nos. 5,165,417, 5,844,997, 6,139,505, 6,394,967 to Raymond Murphy, the inventor herein, disclose multichannel sound recording system based on a multichannel A/D board.

U.S. Pat. No. 4,053,951, to Hudspeth, et al. entitled Data acquisition, storage and display system discloses the device for medical data acquisition including temperature, respiration rate and pulse rate are measured and stored in an acquisition unit incorporating a circulating register for storing data covering many patients.

U.S. Pat. No. 5,701,904 to Simmons, et al., entitled Telemedicine instrumentation pack discloses a portable medical diagnostic apparatus which includes three types of data-gathering instruments: (1) visual instruments (eg, otoscope, ophthalmoscope, rhino-laryngoscope, macro lens and fundus camera); (2) an audio instrument (eg, electronic stethoscope); and (3) data-gathering instruments (eg, pulse oximeter and ECG monitor). The signals are transmitted to a remote site for analysis by medical personnel.

Although these devices fulfill the purpose of multichannel data acquisition they all rely on special data acquisition hardware which makes them expensive and cumbersome. Recording two or more channels of data via the ubiquitous microphone port is advantageous for many reasons. The multichannel sound recording system disclosed herein is based on the existing microphone port and consequently does not require an addition of data acquisition hardware resulting in a cheaper and less cumbersome system.

Further, a highpass filter on the input of the microphone port prevents recording of data below 20 Hz. Many physiological signals below 20 Hz are of great importance, for example EKG and seismocardiogram. The invention disclosed herein uses amplitude modulation of a carrier frequency. The particular carrier frequencies are chosen from frequency range that is unaffected by the microphone port hardware filters. This allows recording of low frequency signals, that is signals below 20 Hz, via the microphone port of the computing device.

### BRIEF SUMMARY OF THE INVENTION

The invention disclosed herein extends the recording frequency range of a microphone port to very low frequencies and allows a plurality of data channels to be transmitted into a computing device via the microphone port using multiple frequency bands. Briefly, amplitude modulation occurs in the hardware using a set of carrier frequencies. The resulting amplitude modulated signals are summed into a composite signal which is transmitted into the microphone port of the computing device. Demodulation occurs in the software. The composite signal can be transmitted to the computing device by wires, by wireless data communications, by a network of computing devices or by a combination of these means.

The stethoscopes are widely used by medical personnel to listen to body sounds. Unfortunately the stethoscopes do not allow recording or visualization of sounds, nor do they allow to easily relate heart sounds to the events of the heart cycle apparent on the EKG. In the preferred embodiment, referred thereafter as “EKG Stethoscope”, the disclosed method is used to simultaneously transmit the audio signal from an electronic stethoscope and the corresponding electrical EKG signal into a computing device via the microphone port of the computing device. In other words, the EKG Stethoscope allows the medical practitioner to perform auscultation and obtain electrocardiogram at the same time. The recording/visualization device could be a personal computer, a PDA, a mobile phone, a land line phone or a voice recorder. The data can be transmitted via wire or wirelessly (for example using Bluetooth technology).

The EKG Stethoscope has the following advantages:

A phonocardiogram can be visualized simultaneously with an electrocardiogram.

Auscultation of heart sounds is greatly facilitated by knowing the event of the heart cycle visualized on the EKG.

Automatic, that is computer based, heart sound analysis is facilitated by identification of events on the electrocardiogram.

The EKG Stethoscope system uses the fact that neither the EKG nor the audio signal requires the full bandwidth of the microphone port (which is 20 Hz to 44,100 Hz). Normally the EKG signal is between 0.5 Hz and 300 Hz, and body sounds are between 20 Hz and 2000 Hz. Therefore, there is sufficient bandwidth to transmit both EKG and sound into the microphone port of the computing device.

In an alternative embodiment physiological data from multiple sensors, such as acoustic pick-up sensors, are transmitted into the computing device via a single microphone port. Similarly, body sounds are limited in bandwidth to 2000 Hz. Therefore, theoretically, up to 11 channels can be modulated and concurrently transmitted into a microphone port with bandwidth of 44,000 Hz.

### BRIEF DESCRIPTION OF THE DRAWING

FIG. 1A is a flow chart of a system for implementing data acquisition from a plurality of data channels via a single microphone port of a computing device;

FIG. 1B is a flow chart of a system for implementing demodulation of a composite signal of FIG. 1A;

FIG. 2 is a block diagram of a system for implementing a preferred embodiment of the present invention;

FIG. 3 is a flow chart of the steps performed in Signal Conditioning and Modulation Circuit of FIG. 2.

FIG. 4 shows overall design of the EKG Stethoscope with EKG electrodes embedded into the chest piece as viewed from the bottom.

FIG. 5 is a data plot of the composite signal (top) separated by filtering into modulated EKG (middle) and audio signal (bottom).

FIG. 6 is a data plot of the amplitude modulated EKG (top), the EKG multiplied by carrier signal (middle) and the EKG lowpass filtered with a cutoff frequency equal to 25 Hz resulting in a clean EKG signal (bottom).

FIG. 7 is a data plot of the composite signal (top) and the recovered signals. The EKG signal is shown in the middle and heart sound is shown in the bottom.

### DETAILED DESCRIPTION OF THE INVENTION

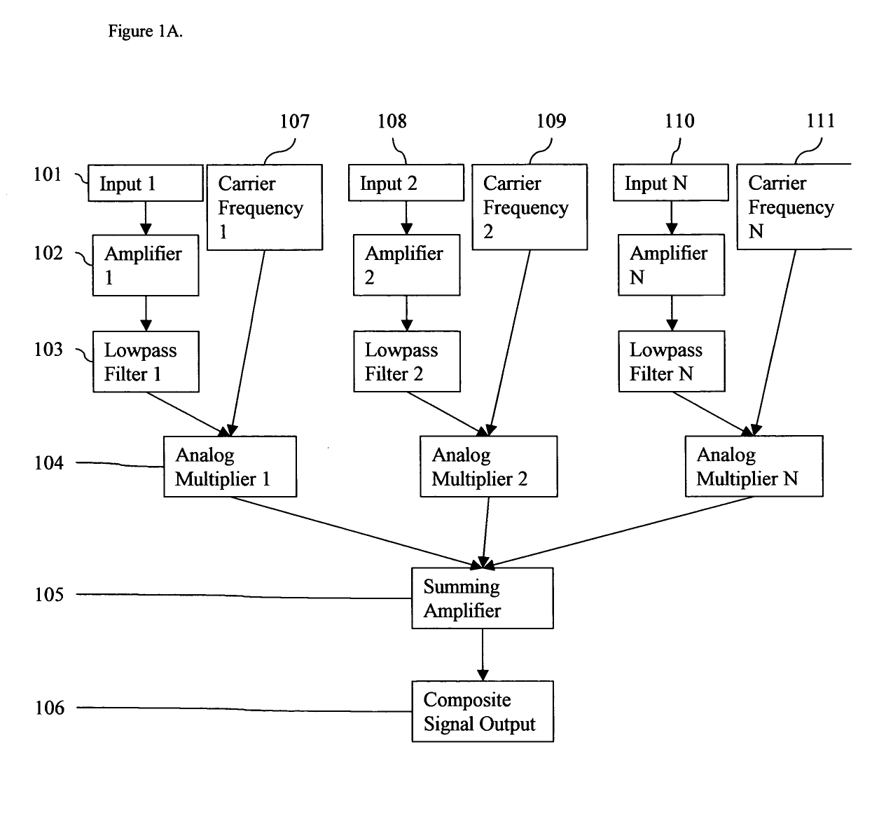

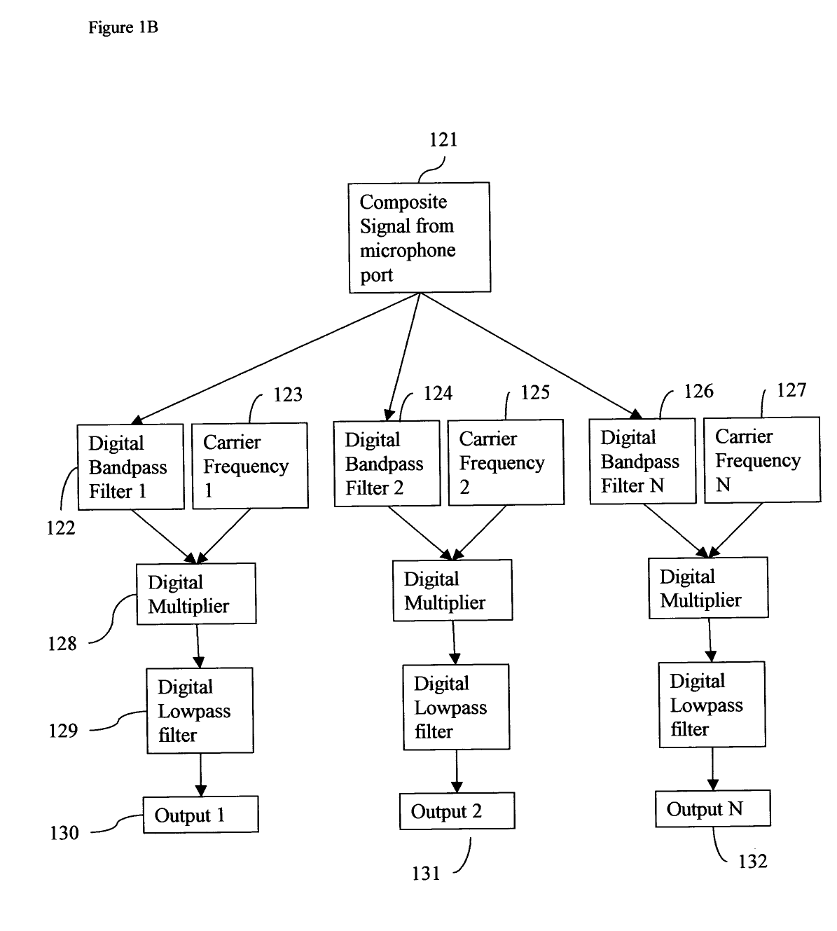

FIG. 1A is a flow chart of a system for implementing data acquisition from a plurality of data channels via a microphone port of a computing device. Input 1 101 is first amplified by an Amplifier 102 and then is filtered by a Lowpass Filter 103 with cutoff frequency of f_{cutoff}. Further the resulting signal is multiplied by the carrier frequency f_{carrier} 107 in an Analog Multiplier 104. The resulting modulated input 1 signal is moved up on the frequency scale to occupy the interval from f_{carrier}−f_{cutoff} to f_{carrier}+f_{cutoff}.

A plurality of inputs from Input 2 108 to Input N 110 can be modulated by the corresponding carrier frequencies 109, 111. All modulated signals are summed by a Summing Amplifier 105 to derive a composite signal output 106. The composite signal can be then transmitted into the microphone port of the computing device via wire or wirelessly.

Consider an 8 channel data acquisition system transmitting data into a standard computer sound card with sampling rate of 44,100 Hz. Each input channel of the 8 channel data acquisition system records data from a sensor with a bandwidth of 0 Hz to 1,000 Hz. All eight lowpass filters can be chosen to have cutoff frequency equal to 1,000 Hz. Eight carrier frequencies can be chosen as follows: f1=2,500 Hz, f2=5,000 Hz, f3=7.500 Hz, f4=10,000 Hz, f5=12,500 Hz, f6=15,000 Hz, f7=17,500 Hz, f8=20,000 Hz. Amplitude modulation allows to distribute 8 data channels over the frequency range of the sound card. The carrier f1 modulated by input 1 occupies interval from 1,500 Hz to 3,500 Hz, the carrier f2 modulated by input 2 occupies interval from 4,000 Hz to 6,000 Hz, . . . , the carrier f8 modulated by input 8 occupies interval from 19,000 Hz to 21,000 Hz. The summing amplifier then sums eight amplitude modulated carrier frequencies into a composite signal.

The composite signal is then transmitted into the microphone port of the computing device via wire or wirelessly.

Inside the computing device the composite signal is demodulated. As long as intervals occupied by modulated signals in the frequency domain are separated, it is possible to recover original signals with no loss. The demodulation flow chart is shown in FIG. 11B. The composite signal 121 is digitized by the computing device sound card. A digital bandpass filter 1 122 is used to separate the frequency band around the carrier frequency 1 123 from f_{carrier}−f_{cutoff} to f_{carrier}+f_{cutoff}. The resulting signal is multiplied by a digitally generated carrier frequency 1 123 in a digital multiplier 128. The resulting signal is filtered by a Digital Lowpass Filter 129. As long as the carrier frequency 123 is equal to the carrier frequency 107 of FIG. 1A, the resulting Output signal 1 130 is equal to the Input 1 101 of FIG. 1A.

Similarly, the composite signal 121 can be broken down into a plurality of frequency bands by digital bandpass filters 2 124 through N 126. Each band is multiplied by the corresponding carrier frequency 125 through 127 and consequently filtered with lowpass filters. The resulting demodulated output signals 2 131 through N 132 are indistinguishable from the corresponding inputs 108 through 110. These outputs can now be recorded, visualized, and analyzed by the computing device.

In the example of the eight channel data acquisition system mentioned above the digital bandpass filter 1 can be a Hamming bandpass filter with 512 taps and pass band from 1,500 Hz to 3,500 Hz; the digital bandpass filter 2 can be a Hamming bandpass filter with 512 taps and pass band from 4,000 Hz to 6,000 Hz; . . . ; the digital bandpass filter 8 can be a Hamming Window bandpass filter with 512 taps and pass band from 19,000 Hz to 21,000 Hz. Further each channel is digitally multiplied by the corresponding carrier frequency. The output of the digital bandpass filter 1 is multiplied by the carrier frequency 1 equal to 2,500 Hz; the output of the digital bandpass filter 2 is multiplied by the carrier frequency 2 equal to 5,000 Hz, . . . ; the output of the digital bandpass filter 8 is multiplied by the carrier frequency 8 equal to 20,000 Hz. Further the result of multiplication is filtered by a digital lowpass filter. All digital lowpass filters can be Hamming Window lowpass filters with 512 taps and pass band between 0 Hz and 1000 Hz.

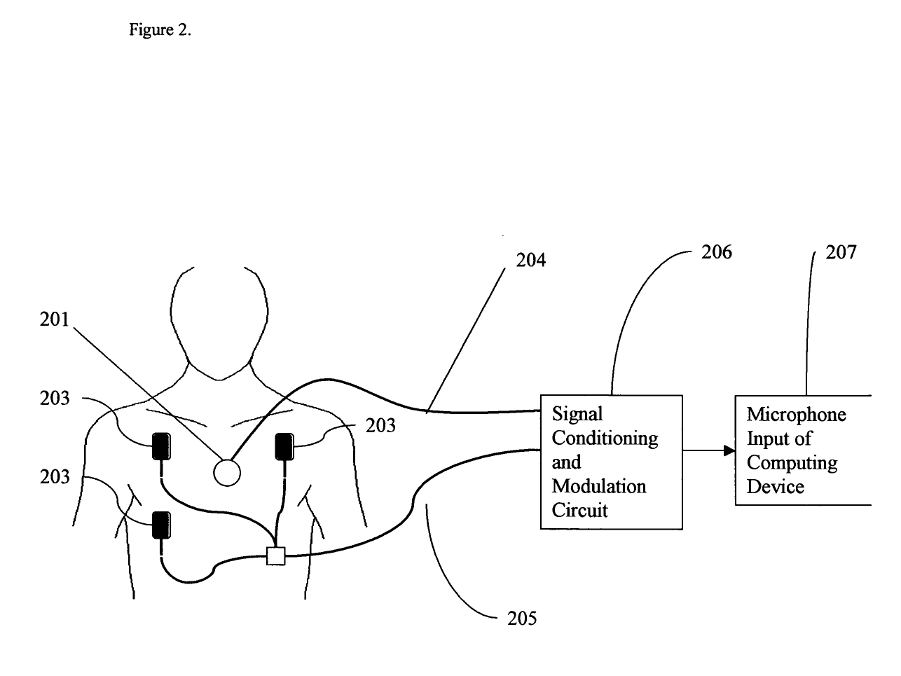

FIG. 2 shows a block diagram of a system for implementing a preferred embodiment of the present invention, the EKG Stethoscope. The chest piece 201 picks up an acoustic signal from the body, converts the acoustic energy into an electrical signal and than transmits the signal via wire or wirelessly 204 into a Signal Conditioning and Modulation Box 206. Further the electrocardiographic signal from the patient's skin is picked up by EKG electrodes 203 and transmitted via wire or wirelessly 205 into a Signal Conditioning and Modulation Box 206. The composite signal from the Signal Conditioning and Modulation Box 206 is transmitted to the Microphone Port of the computing device 207.

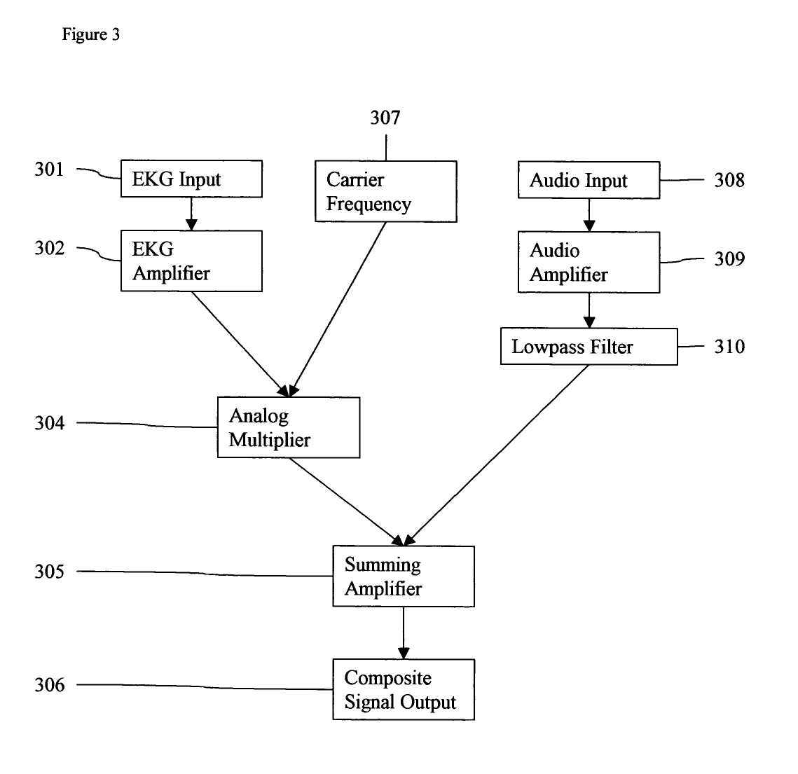

FIG. 3 describes the flow chart of the steps performed in the Signal Conditioning and Modulation Box 206 of FIG. 2. The EKG input 301 from EKG electrodes placed on patient's skin is amplified by a standard EKG amplifier 302 with a gain of 1V/mV. Further it is filtered by a bandpass filter 0.5 to 120 Hz and 60 Hz notch filter (−23 dB). The resulting amplified and filtered EKG is multiplied by the carrier signal 307 with frequency 3,000 Hz in the Analog Multiplier 304. Note that the resulting modulated EKG signal is located in the frequency band centered around the carrier frequency, that is between 2,700 Hz and 3,300 Hz.

The audio input 308 from sound pickup placed on the patient's skin is amplified by the Audio Amplifier 309 and filtered by a Lowpass Filter 310 with a cutoff frequency of 2,000 Hz. The modulated EKG signal is then summed with the amplified and filtered audio signal by the Summing amplifier 305. The resulting composite signal output 306 is transmitted via wire or wirelessly into the computing device.

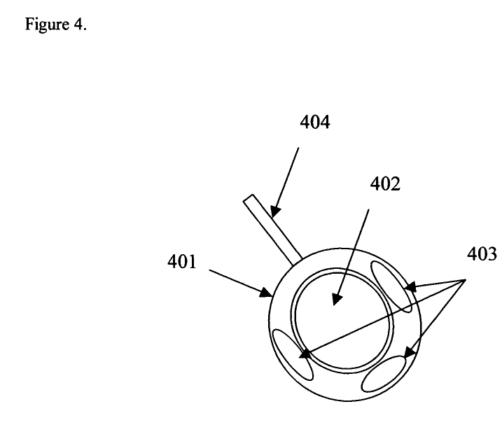

FIG. 4 shows the overall design of the EKG Stethoscope with three EKG electrodes 403 mounted on the chest piece 401 around the diaphragm 402. The physician can move chest piece around the chest to collect data at different sites. The suitable EKG electrodes can be made of electroconductive material and have an area of 1 cm². The sound amplification can be either electronic via wire or acoustic via tubing 404. The suitable microphone for the electronic sound amplification can be omnidirectional electret microphone embedded into the chest piece. The EKG Stethoscope allows a medical practitioner to avoid application of separate EKG electrodes. The result is a faster and less cumbersome procedure.

The computing device of the EKG Stethoscope can be a PDA such as Compaq iPAQ5450 Pocket PC. The composite signal 306 of FIG. 3 is transmitted to the PDA's microphone input port. The transmission can be via the wire connected to an external 3.5 mm microphone jack or wirelessly via bluetooth headset protocol. No modification or special hardware is required with iPAQ5450. The PDA can be programmed to conduct the demodulation of the composite signal. The PDA can display the results of demodulation on its screen and store the data for later retrieval/transfer. Also, the PDA can be programmed to perform the automatic analysis of the EKG and acoustic signals.

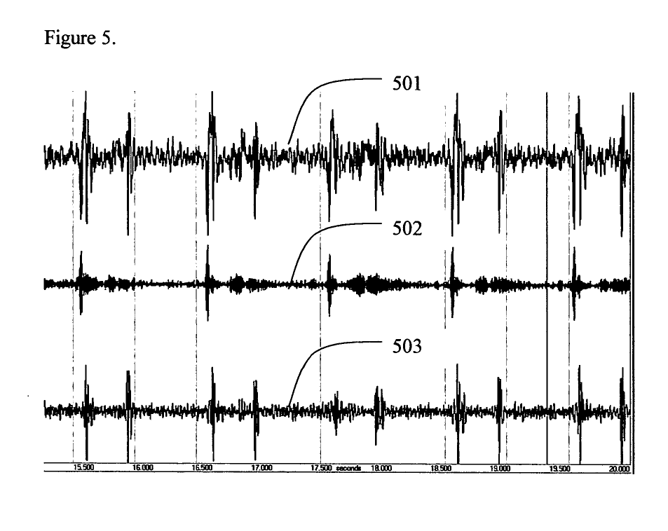

Inside the computing device the composite signal 306 of FIG. 3 is demodulated into an EKG and audio signals for further recording, visualization, and analysis. FIG. 5 is a data plot of a composite signal 501 recorded from a subject. The composite signal 501 is first filtered by a lowpass filter with cutoff frequency of 2000 Hz. The resulting signal is a pure audio signal 503, FIG. 5. Further, the composite signal 501 is filtered by a bandpass filter with cutoff frequencies of 2700 Hz and 3300 Hz. The resulting signal is the modulated EKG signal 502.

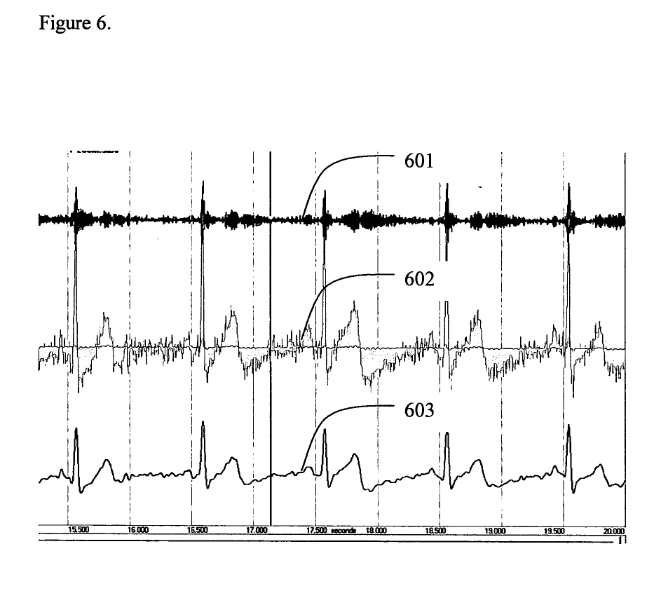

FIG. 6 shows the process of demodulation of the EKG signal 502 of FIG. 5. The modulated EKG signal 601 is multiplied by the carrier frequency. The result of digital multiplication is the signal marked 602. Further, the signal 602 is filtered by a lowpass filter with a cutoff frequency of 25 Hz. The resulting signal is a clean EKG signal 603.

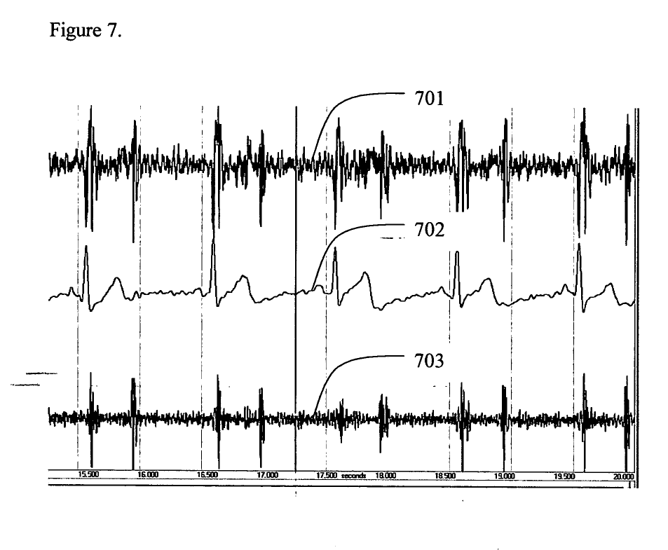

FIG. 7 is a data plot of the composite signal 701, same as 501 of FIG. 5, and the demodulated EKG 702, same as 603 of FIG. 6, and sound signal 703, same as 503 of FIG. 5, shown in the stack mode.

We claim:

1. A method of wired or wireless physiological data acquisition via sound input port of a computing device using amplitude modulation of data with one or more carrier frequencies.

2. The computing device of claim 1 selected from a group consisting of a desktop computer, a notebook, a tablet PC, a PDA, a mobile phone, a land line phone, a tape recorder, and a digital voice recorder.

3. The computing device of claim 1 transmitting data to a secondary computing device, such as a server either via wire or wirelessly.

4. The data of claim 1 including a plurality of channels modulated by multiple carrier frequencies.

5. The carrier frequencies of claim 1 distributed over the permissible sound port frequency range in such a manner that neither frequency band overlaps with any other frequency band.

6. The carrier frequencies of claim 1 supplied by the audio output of the computing device or generated by circuitry outside of the computing device.

7. The sound input port of claim 1 wherein sound input port is a microphone port, a line port, or the wireless sound port of a computing device.

8. The wireless sound port of claim 7 wherein wireless protocol selected from a group consisting of a bluetooth protocol and a Wi-Fi protocol.

9. The bluetooth protocol of claim 8 wherein a headset profile is used to transmit data to and from the computing device.

10. The physiological data acquisition system using said amplitude modulation method of claim 1 to transmit multiple cannels of physiological data to said microphone port of said computing device.

11. The computing device of claim 1 wherein the demodulation of the composite signal by software occurs in real time.

12. The EKG Stethoscope using said amplitude modulation method of claim 1 comprised of:
(a) a stethoscope,
(b) an electrocardiograph, and
(c) EKG electrodes,
whereby a medical practitioner is enabled perform simultaneous auscultation and electrocardiography.

13. The EKG Stethoscope of claim 12 wherein the EKG is modulated by a carrier frequency and added to an audio signal resulting in a composite signal that is transmitted to the sound port of a computing device.

14. The EKG Stethoscope of claim 12 visualizing both phonocardiogram and EKG concurrently on the screen of the computing device in the stack mode or superimposed.

15. The EKG electrodes of claim 12 located on the chest piece to simplify application of said EKG Stethoscope on patients.

16. The EKG electrodes of claim 12 attached to the subject's skin connected to the said EKG Stethoscope via standard wired EKG leads.

17. The EKG Stethoscope of claim 12 having means for visualizing the EKG and audio waveform on a read-out display located on the chest piece.

18. The EKG Stethoscope of claim 12 having means to signal the operator events of the EKG cycle, whereby said event will include the QRS complex, which corresponds to the start of systole, the P-wave, which corresponds to the start of atrial depolarization, and T-wave, which corresponds to the start of diastole.

19. The EKG Stethoscope of claim 12 having means for transmitting sounds from the chest piece to the operators ears.

20. The EKG Stethoscope of claim 12 having the chest piece mounted on a computing device, such as a PDA.

21. The EKG Stethoscope of claim 12 incorporating means for automatic identification of respiratory cycle, automatic identification of events on EKG, and automatic identification of heart sounds components.

22. The physiological data acquisition system using said amplitude modulation method of claim 1 to transmit sound recordings from 2 or more sound pick-up sensors into said sound input port of the computing device whereby each channel is modulated by its own carrier frequency.
